(19) United States
(12) Patent Application Publication
(10) Pub. No.: US 2014/0170089 A1
(43) Pub. Date: Jun. 19, 2014

(54) PHOTOPROTECTIVE EYE CREAM AND METHODS

(71) Applicant: Holly E. THAGGARD, San Antonio, TX (US)

(72) Inventor: Holly E. THAGGARD, San Antonio, TX (US)

(73) Assignee: Taylor James, LLC, San Antonio, TX (US)

(21) Appl. No.: 14/107,409

(22) Filed: Dec. 16, 2013

Related U.S. Application Data
(60) Provisional application No. 61/738,291, filed on Dec. 17, 2012.

(51) Int. Cl. A61K 8/64; A61K 8/25; A61Q 19/00; A61K 8/37; A61Q 19/08; A61Q 17/04; A61K 8/27

(52) U.S. Cl. CPC A61K 8/645; A61Q 17/04; A61K 8/25; A61K 8/27; A61K 8/37; A61Q 19/08; A61Q 19/00; A61Q 19/007
USPC 424/59

(57) ABSTRACT

Photoprotectant cosmetic compositions adapted to application on a user's skin that include a sunscreen component comprising at least two sun-blocking components, each present in an amount sufficient to collectively provide ultraviolet A and B protection with an SPF of at least about 10; a mica component including mica in an amount sufficient to reduce or eliminate at least the appearance of dark skin adjacent an eye; and an oat peptide component in an amount sufficient to reduce or eliminate at least the appearance of fine lines and wrinkles on the user's skin. Methods of improving the aesthetic appearance of skin with such compositions are also included.

### CROSS-REFERENCE TO RELATED APPLICATION

This application claims the benefit of and priority to U.S. Provisional Application No. 61/738,291, filed Dec. 17, 2012, the contents of which is hereby incorporated herein in its entirety by express reference thereto.

### TECHNICAL FIELD

The invention is directed to a photoprotective cosmetic composition for topical application that includes: (a) a sunscreen including octinoxate and zinc oxide, (b) a mica component, and (c) an oat peptide component. The invention further relates to a method of reducing the appearance of fine lines and wrinkles around the eye, increasing skin hydration or moisturization, or a combination thereof, by topically applying the composition to the skin around the eye, and to methods of photoprotecting the skin from the damaging effects of UV radiation by topically applying the composition to the skin.

### BACKGROUND

The skin around the eye is particularly susceptible to aging and the effects of environmental insults, which can lead to eye pouches, black eye circles, crow's feet and the like. While all areas of the skin can experience wrinkles, irritation, and redness, the area under the eye has particular problems, not associated with other part of the skin. In particular, the area around the eye is susceptible to wrinkling and dryness due to the lesser amount of oil glands compared to the rest of the face or other areas of skin. Also, the skin of the eyelid is extremely thin, the thinnest in the human body, and, thus, readily susceptible to damage, such as from the sun.

There is a need in the art for formulations that minimize or reduce the effects of aging and environmental insults on the skin around the eye.

### SUMMARY

The present disclosure encompasses an invention directed to photoprotectant cosmetic compositions including a photoprotectant cosmetic composition adapted to application on a user's skin that includes a sunscreen component including: (i) octinoxate; and (ii) zinc oxide, each present in an amount sufficient to collectively provide ultraviolet A and B protection with an SPF of at least about 10, a mica component including mica in an amount sufficient to reduce or eliminate the appearance of dark skin adjacent an eye, and an oat peptide component in an amount sufficient to reduce or eliminate at least the appearance of fine lines and wrinkles on the user's skin.

In one embodiment, the photoprotectant cosmetic composition is formulated as a cream. In another embodiment, the photoprotectant cosmetic composition is formulated as an emulsion. In a preferred embodiment, the sufficient amount of octinoxate is about 5% to about 15% by weight of the composition and the zinc oxide is about 5% to about 25% by weight of the composition, the sufficient amount of mica component is about 0.5% to about 1.5% by weight of the composition, and the sufficient amount of the oat peptide component is about 0.02% to about 0.15% by weight of the composition. In a more preferred embodiment, the octinoxate is present in an amount from about 8% to about 15% by weight of the composition, the zinc oxide is present in an amount from about 5% to about 25% by weight of the composition, the mica component is present in an amount from about 0.8% to about 1.2% by weight of the composition, and the oat peptide component is present in the composition in an amount from about 0.05% to about 0.1% by weight of the composition.

In another embodiment, the above-noted photoprotectant cosmetic compositions do not include octocrylene or amiloxate, or both. In another embodiment, the compositions of the present disclosure are at least substantially free of octocrylene or amiloxate, or both. In one embodiment, the photoprotectant cosmetic compositions have an SPF from about 10 to about 50. In a preferred embodiment, the SPF of the composition is from about 35 to about 50. In one more preferred embodiment, the SPF of the composition is from about 35 to 40. In yet another embodiment, the invention encompasses a photoprotectant cosmetic composition adapted to application on a user's skin including a sunscreen component containing only (i) octinoxate; and (ii) zinc oxide, each present in an amount sufficient to collectively provide ultraviolet A and B protection with an SPF of at least about 10; along with a mica component including mica in an amount sufficient to reduce or eliminate at least the appearance of dark skin adjacent an eye; and an oat peptide component in an amount sufficient to reduce or eliminate at least the appearance of fine lines and wrinkles on the user's skin, where the composition as a whole does not include a detectable amount of octocrylene and amiloxate.

In one preferred embodiment, the photoprotectant cosmetic composition further includes a stearate component, an alcohol component, and a formulating agent. The formulating agent preferably includes cyclopentasiloxane, alkyl benzoate, potassium cetyl phosphate, an adipic acid/diethylene glycol/glycerin crosspolymer, or isohexadecane, or a combination of one or more thereof. In another preferred embodiment, the stearate component includes ethylhexyl stearate, glyceryl stearate, or PEG-100 stearate, or a combination thereof, the alcohol component includes a diol, a glycol, or cetearyl alcohol or a combination thereof, and the alkyl benzoate is a C₁₂₋₁₅ alkyl benzoate. In a more preferred embodiment, the stearate component includes a combination of ethylhexyl stearate, glyceryl stearate, and PEG-100 stearate, the alcohol includes a combination of propanediol, butylene glycol, and cetearyl alcohol, and the formulating agent includes a combination of cyclopentasiloxane, alkyl benzoate, potassium cetyl phosphate, an adipic acid/diethylene glycol/glycerin crosspolymer, and isohexadecane. In an exemplary embodiment, the stearate component is present in an amount of from about 6% to about 7.5% by weight of the composition, the alcohol is present in an amount of from about 8% to about 9% by weight of the composition, and the formulating agent is present in an amount of from about 5.5% to about 11% by weight of the composition.

In another aspect, the invention encompasses methods of improving the aesthetic appearance of skin, which includes topically applying a coating of any of the compositions disclosed herein to the skin. The improvement in aesthetic appearance, in one embodiment, includes an increase in skin hydration, reduction in appearance of fine lines and wrinkles in an eye area, decrease in the number of fine lines of a crow feet's area, improvement in skin texture or smoothness of fine lines of a crow feet's area, or a combination thereof. In another embodiment, the improvement in aesthetic appearance is an increase in skin hydration, and topical application effects an increase in skin hydration of greater than 10% from baseline. In a separate embodiment, the improvement in aesthetic appearance is a reduction in appearance of fine lines and wrinkles in an eye area, and topical application effects a reduction of greater than 3% from baseline. In another embodiment, the improvement in aesthetic appearance is an improvement in skin texture or smoothness of fine lines of a crow feet's area, and topical application effects an increase of smoothness of greater than 5% from baseline. In yet another embodiment, the improvement in aesthetic appearance is a decrease in the number of fine lines of a crow feet's area, and topical application effects a decrease in the number of fines lines of greater than 20% from baseline. In various embodiments, the topical applying occurs once or twice daily for about four to eight weeks.

### BRIEF DESCRIPTION OF THE DRAWINGS

The present disclosure is best understood from the following detailed description when read with the accompanying figures. It is emphasized that, in accordance with the standard practice in the industry, various features may not be drawn to scale. In fact, the dimensions of the various features may be arbitrarily increased or reduced for clarity of discussion.

FIG. 1 is a schematic representation of the test sites where a composition according to an embodiment of the present disclosure was applied.

### DETAILED DESCRIPTION OF THE PREFERRED EMBODIMENTS

The compositions and methods of the present disclosure are directed to improving skin moisturization and hydration, decreasing the appearance of fine lines and wrinkles around the eye, decreasing the number of fine lines in the crow's feet area of the eye, and improving the skin texture/smoothness of fine lines in the crow's feet area of the eye. As used herein, the term “wrinkle” refers to coarse or deep wrinkling. Coarse wrinkling refers to deep furrows, particularly deep lines/wrinkles on the face and around the eyes. “Fine lines” refer to superficial lines on the skin surface that are not deep lines. The “crow's feet area” refers to the skin of the sides of the eye.

The photoprotectant cosmetic compositions of the invention disclosed herein include a sunscreen component that includes (i) octinoxate; and (ii) zinc oxide in amounts effective to provide at least an SPF 8, preferably at least an SPF 10 to about 50, and more preferably an SPF of about 35 to about 45, along with a mica component that includes mica in an amount sufficient to reduce or eliminate an appearance of dark skin and an oat peptide component in an amount sufficient to reduce or eliminate at least the appearance of fine lines and wrinkles on the user's skin. These compositions are preferably formulated as a cream or lotion of suitable viscosity to be applied to and retained on the skin, particularly on the face, and preferably around the eyes. The composition is typically an emulsion to provide a suitable texture.

Octinoxate is chemically known as octyl methoxycinnamate. The structure of octinoxate is:

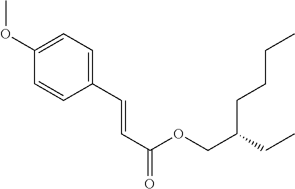 [SMILES: CCCC[C@H](CC)COC(=O)/C=C/c1ccc(OC)cc1]

and zinc oxide has the chemical formula (ZnO). Typically, the octinoxate is present in the composition in an amount ranging from about 5% to about 15% by weight of the composition, preferably about 8% to about 15% by weight of the composition. In one embodiment, the octinoxate is present in the composition in an amount from about 8% to about 10% by weight of the composition. In one embodiment, the octinoxate is present in the composition in an amount of about 10%.

Typically, the zinc oxide is present in the composition in an amount ranging from about 5% to about 25% by weight of the composition, preferably about 5% to about 21% by weight of the composition. In one embodiment, the zinc oxide is present in the composition in an amount ranging from about 7.5% to about 15% by weight of the composition. In various embodiments, the zinc oxide is present in the composition in amounts of about 9%, 10%, 15%, 20%, and 21%, each by total weight of the composition.

The term sunscreen, as used herein, is a composition that when applied to the skin forms a coating, or film, where applied that protects the skin against the damaging effects of ultra-violet (UV) exposure. The effectiveness of a sun screen, i.e., its ability to block UV radiation is typically expressed as a sun protection factor or “SPF” rating. Sunscreens protect the skin by absorbing UV radiation before it can interact with and damage the skin, however, their efficacy is often limited in time. Formulation of the sunscreen can affect the length of time a photoprotectant effect is provided.

Typically, the mica component includes a plurality of mica particles present in the composition in an amount ranging from about 0.5% to about 1.5% by weight of the composition, preferably about 0.75% to about 1.35% by weight of the composition, more preferably about 0.8% to about 1.2% by weight of the composition by weight of the composition. An exemplary embodiment includes mica particles in an amount of about 0.90% to 1.10% of the total composition. In one embodiment, these weight percentages are of the mica itself, while in another they relate to the coated mica particles. Mica is a silicate mineral. Typically, the mica is provided in the composition as finely-ground small particles, i.e., as a powder. The particles may be of substantially uniform size within a narrow band of size ranges, such as within about 20%, preferably within about 10% size difference between the largest and smallest particles. In another embodiment, the mica particles may be of different groupings by size, while in another embodiment, the mica particle size differs across a size range of at least about 30%, preferably at least about 50% by size of the largest and smallest particles. Preferably, the mica particles can also be coated with any other suitable coating material, for example, to minimize interaction with other components, to enhance visual appeal, to increase stability of the mica particles in the sunscreen compositions, or a combination thereof, etc.

The composition may include a peptide component, such as oat peptide, apple peptide, grape peptide, and combinations of one or more of each type thereof. Preferably, the composition includes an oat peptide component present in an amount ranging from about 0.02% to about 0.15% by weight of the composition, preferably about 0.03% to about 0.12% by weight of the composition, more preferably about 0.05% to about 0.1% by weight of the composition. The oat peptide component may include one or more different types of oat peptides. In one preferred embodiment, the oat peptide component includes oat peptide obtained or extracted from the seeds of the oat, grape, or apple. Preferably, the oat peptide component is included in the compositions and is obtained from the avena sativa plant. The peptide component may also be provided in aqueous solution or suspension form, such as in a water-base.

Without being bound by theory, it is believed that certain combinations of sunscreen components may have deleterious effects on the skin, on the photoprotectant effects of the composition, on the stability of the composition, etc. Thus, in one embodiment, the composition does not include one or more of octocrylene or amiloxate. In another preferred embodiment, the composition does not include octocrylene and amiloxate. In one embodiment, the composition is at least substantially free of octocrylene or amiloxate, or both. By “substantially free” is meant that less than 3% by weight is present of octocrylene or amiloxate, or both, preferably less than 1% by weight, and more preferably less than 0.5% by weight. In yet another preferred embodiment, the composition does not include any other sunscreen component that modifies the SPF factor of the composition in a meaningful manner.

The photoprotectant cosmetic compositions typically have an SPF rating of at least 5, preferably at least about 15 and more preferably at least about 30. In one embodiment, the SPF rating is above about 20. In one embodiment, the SPF rating is above about 22. In one embodiment, the SPF rating is at least about 30. In one embodiment, the SPF rating is at least about 35. In one embodiment, the SPF rating is at least about 40. In one embodiment, the SPF rating is at least about 45. A preferred SPF rating of the composition is from about 30 to about 50, and another preferred SPF rating is from about 35 to 45. SPF rating is determined using FDA approved method. See U.S. Food and Drug Administration. Labeling and Effectiveness Testing; Sunscreen Drug Products for Over-the-Counter Human Use; Final Rule; 21 CFR Parts 201 and 310. Federal Register, Vol. 76, No. 117, Jun. 17, 2011. pp. 35660-35665 (“FDA Final Rule”).

In various preferred embodiments, the compositions further include of the following components in an aqueous mixture: a stearate component (including one or more of ethylhexyl stearate, glyceryl stearate, PEG-100 stearate, or combinations thereof), cyclopentasiloxane, an alcohol (e.g., a diol, such as propanediol; a glycol, such as butylene glycol; cetearyl alcohol; and combinations thereof), C₁₂₋₁₅ alkyl benzoate, potassium cetyl phosphate, an adipic acid/diethylene glycol/glycerin crosspolymer, isohexadecane, and combinations of one or more thereof.

In some embodiments, the stearate component is present in an amount ranging from about 3.0% to about 10.0% by weight of the composition, preferably about 4.5% to about 8.5%, more preferably about 6.0% to about 7.5% by weight of the composition. In one embodiment, the stearate may include any one or more suitable stearates available to those of ordinary skill in the art. Exemplary stearates include glyceryl stearate, a C₁-C₁₀ alkyl stearate, a glycol stearate, or any combination thereof. In a preferred embodiment, the alkyl stearate includes ethylhexyl stearate, the glycol stearate includes PEG stearate, or both. Cyclopentasiloxane may be present in an amount ranging from about 2.5% to about 7.0% by weight of the composition, preferably about 3.0% to about 6.5%, and more preferably about 4.0% to about 5.0%. The alcohol component may be present in an amount ranging from about 5.5% to about 12.0% by weight of the composition, more preferably about 6.5% to about 10.0%, and most preferably about 8.0% to about 9.0%. In one embodiment the alcohol may include any one or more suitable alcohols available to those of ordinary skill in the art, with exemplary alcohols including propanediol, butylene glycol, and one or more C₁₀-C₂₀ alcohols, or any combination thereof. The alcohol is preferably a mixture of C₁₃-C₁₇ alcohols, such as cetearyl alcohol (e.g., formed from a mixture of cetyl and stearyl alcohols). The C₁₂₋₁₅ alkyl benzoate component may be present in an amount ranging from about 0.5% to about 5.0% by weight of the composition, more preferably from about 1.5% to about 4.0%, and most preferably from about 2.0% to about 3.0%. Potassium cetyl phosphate may be present in an amount ranging from about 0.5% to about 4.5% by weight of the composition, more preferably from about 1.0% to about 4.0%, and most preferably from about 2.0% to about 3.5%. An adipic acid/diethylene glycol/glycerin crosspolymer may be present in an amount of about 0.25% to about 4.0% by weight of the composition, more preferably from about 0.75% to about 1.75%, and most preferably from about 1.0% to about 2.5%. The isohexadecane component may be present in an amount ranging from about 0.3% to about 3.5% by weight of the composition, more preferably from about 0.8% to about 2.5%, and most preferably from about 1.4% to about 2.0%.

The compositions herein can include a variety of other components known to be included in skin care compositions. The CTFA Cosmetic Ingredient Handbook, Seventh Edition, 1997 and the Eighth Edition, 2000, which is incorporated herein by reference in its entirety, describes a wide variety of cosmetic and pharmaceutical ingredients commonly used in skin care compositions, which are suitable for use in the compositions of the present invention. Illustrative examples of the functional classes of these ingredients include, but are not limited to, one or more of the following: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, SPF boosters, waterproofing agents, and viscosity increasing agents (aqueous and nonaqueous).

The compositions of the invention are prepared by admixing the components using procedures well known to formulators of cosmetic compositions. Preferably, the composition is formulated as a cream or lotion. The composition is preferably formulated to be applied as a coating or film on the skin, preferably on the face, and more preferably as an eye cream to be applied around the eyes. Preferably, the composition is formulated to include, or to be, an emulsion.

The invention further relates to a method of reducing the appearance of fine lines and wrinkles around the eye of a subject, increasing skin hydration, decreasing the number of fine lines of the crow's feet area, improvement in skin texture/smoothness of fine lines of the crow's feet area, or a combination thereof. The method involves topically applying the composition to the skin around the eye.

Without wishing to be bound by theory, it is believed that the sunscreen component, the mica component, and the oat protein component act synergistically to both provide a sufficient photoprotectant effect for at least about 80 minutes, preferably at least about 120 minutes after application, while also better reducing the appearance of fine lines and/or wrinkles around the eye of a user than would be predicted from the effects of each component acting individually, i.e., they act synergistically. Preferably, this includes the compositions achieving an SPF of at least about 35 to about 50 in a cosmetic eye cream formulation.

The invention further relates to methods of photoprotecting the skin of a subject from the damaging effects of UV radiation. The method involves topically applying the composition to the skin of the user. In one embodiment, the method involves applying the composition to the skin around the eye region of the user in a coating or film sufficient to achieve the synergistic results of photoprotection and reduction in appearance of fine lines and/or wrinkles on the skin it is disposed over.

### EXAMPLES

The following examples are set forth to assist in understanding the invention and should not be construed as specifically limiting the invention described and claimed herein.

### Example 1

### Comparative SPF Test of a Composition According to One Embodiment

A composition was prepared containing the following components:* * Components listed below the line are present in an amount of less than 1% by weight. Other components are listed in descending order of predominance.
Octinoxate in an amount of 7.5% (by weight)
Zinc Oxide in an amount of 10.1% (by weight)
Water
Ethylhexyl Stearate
Cyclopentasiloxane
Propanediol
Butylene Glycol
C₁₂₋₁₅ Alkyl Benzoate
Potassium Cetyl Phosphate
Adipic Acid/Diethylene Glycol/Glycerin Crosspolymer
Isohexadecane
Cetearyl Alcohol
Glyceryl Stearate
PEG-100 Stearate
Mica
Phenoxyethanol
Hydroxyethyl Acrylate/Sodium Acryloyldimethyl Taurate Copolymer
Imperata Cylindrica Root Extract
Titanium Dioxide
Ceteareth-20
Squalane
Glycerin
Triethoxycaprylylsilane
Xanthan Gum
Helianthus Annuus (Sunflower) Seed Oil
Polysorbate 60
Trisodium Ethylenediamine Disuccinate
Chlorphenesin
Citric Acid
Dimethicone
Dipotassium Glycyrrhizate
Ethylhexylglycerin
Oleth-3 Phosphate
PEG-8
Punica Granatum Seed Oil
Carthamus Tinctorius (Safflower) Seed Oil
Avena Sativa (Oat) Peptide
Silica
Camellia Sinensis Leaf Extract
Carbomer
Olea Europaea (Olive) Fruit Extract
Euterpe Oleracea Fruit Extract
Methylisothiazolinone
Tocotrienols
Lauroyl Lysine

The sun protection factor (“SPF”) of the composition was measured for the composition of Example 1 according to FDA requirements (See FDA Final Rule).

Summary:

On the first day of the study each subject received a series of UV doses from a xenon arc solar simulator to an unprotected site on the mid-back. The solar simulator was a single-port xenon arc lamp with a 1 mm WG320 UVC blocking filter, a 1 mm UG-11 visible and infrared blocking filter and a heat rejecting dichroic mirror (Model 16S, Solar Light Co., Philadelphia). On the second day the minimal erythema dose (MED) was determined as the lowest UV dose which produced perceptible erythema with clearly defined borders. Then 100 mg of the test product and 100 mg of a 7% Padimate-O/3% Oxybenzone standard were applied to separate, adjacent 50 cm² areas of the mid-back (standard sunscreen provided by Cosmetech Laboratories, Inc., Fairfield, N.J.). Each sunscreen-protected site was divided into five subsite test areas that were at least 0.5 cm² in area for UV exposures.

The test product had an SPF of about 40. The 7% Padimate-O/3% Oxybenzone standard sunscreen had an SPF of 16.3. After a 15-minute drying period UV doses ranging from 0.76 to 1.32 times the product of the MED and 40 were administered to the test sunscreen-protected area and UV doses ranging from 0.76 to 1.32 times the product of the MED and 16.3 were administered to the standard sunscreen protected areas. A series of UV doses were also administered to a second unprotected site. On the third day the MED was determined for the sunscreen-protected sites (MEDp) and the unprotected sites (MEDu). The SPF of each sunscreen was calculated as the ratio of the MEDp for each sunscreen-protected site to the Final MEDu. The Final MEDu used for SPF computation was the Repeat MEDu for all of the subjects tested.

According to the FDA Final Rule, the labeled SPF must be calculated as follows: SPF values for individual subjects (SPFi) will be calculated as: SPFi=MEDp/MEDu. The mean SPF and standard deviation (SD) will be calculated from valid SPFi values. The Standard Error (SE) will be calculated as: SE=SD/√n, where n equals the number of subjects who provided valid test results. The t value from Student's t distribution table corresponding to the upper 5% point with n−1 degrees of freedom will be obtained. The labeled SPF value will be determined as the largest whole number less than the following calculation:

Labeled SPF=Mean SPF−(t*SE)

In order for the SPF determination of the test product to be valid, the SPF value of the 7% Padimate-O and 3% Oxybenzone Standard should fall within the standard deviation range of the expected SPF (i.e., 16.3±3.43 or 12.87 to 19.73).

Results: Ten subjects, 3 men and 7 women, who provided written, informed consent, completed the study. Subjects included 5 with skin type II (always burns easily; tans minimally (sensitive)) and 5 with skin type III (burns moderately; tans gradually (normal)). Ages ranged from 19 to 64 years and the mean age was 46.80 (n=10, SD=13.97). Subject demographic and static SPF results are listed in Table 1.

The mean static SPF of the test product, was 39.21 (n=10, SD=3.73). The mean SPF−(t*SE), rounded down to the nearest whole number was 37. The mean SPF of the 7% Padimate-O/3% Oxybenzone standard was 16.95 (n=10, SD=1.25). The SPF value of the 7% Padimate-O/3% Oxybenzone standard was within the required range.

### Protocol Deviations:

Protocol Deviations were reported for two subjects. The Final Evaluation of the test product was performed at 24:01 on Subject 04 and UV doses to the standard sunscreen were incorrectly calculated (used ISO standard doses and intervals) for Subject 09. In the opinion of the Investigator these Protocol Deviations did not affect study results.

### Enrollment:

All of the subjects enrolled in the study completed all study procedures.

### Adverse Events:

No Adverse Events were reported.

### Conclusion:

Ten subjects completed the test. The mean static SPF of the composition of Example 1 was 39.21 (n=10, SD=3.73). The composition meets FDA Final Rule requirements for labeling the eye cream composition disclosed herein as Static SPF 37.

TABLE 1
Subject Demographic and Static SPF Results for Composition of Example 1 and 7% Padimate/3% Oxybenzone Standard

| | | | | | | | | | | | 7% Padimate-O/ | |
|---|---|---|---|---|---|---|---|---|---|---|---|---|
| | | | | | | | | | | | 3% Oxybenzone | |
| | | | | | | | | | Example 1 | | Standard | |
| Tech | | SRL | | | | Skin | MED_{UI} (eff | MED_{UR} (eff | tpMEDₚ (eff | | ssMEDₚ | |
| Initials | Subject # | ID# | Initials | Age | Sex | Type | J/m²) | J/m²) | J/m²) | SPF | (eff J/m²) | SPF |
| AOS | 01 | 2070 | WJL | 62 | M | II | 173.78 | 178.67 | 6947.11 | 38.88 | 3262.72 | 18.26 |
| JL | 02 | 2745 | CHL | 53 | F | II | 122.67 | 125.00 | 4907.50 | 39.26 | 2003.22 | 16.03 |
| MPB | 03 | 2237 | MBL | 29 | M | III | 157.50 | 152.50 | 7242.67 | 47.49 | 2956.33 | 19.39 |
| MPB | 04 | 1626 | MJH | 54 | F | III | 142.22 | 146.00 | 4945.50 | 33.87 | 2315.33 | 15.86 |
| JMW | 05 | 2304 | BDR | 64 | F | III | 197.89 | 197.22 | 7915.28 | 40.13 | 3224.00 | 16.35 |
| AOS | 06 | 2892 | GAM | 51 | M | II | 184.00 | 187.89 | 7360.00 | 39.17 | 3451.22 | 18.37 |
| JMW | 07 | 2073 | PSP | 49 | F | II | 175.06 | 178.67 | 7004.00 | 39.20 | 2853.28 | 15.97 |
| JMW | 08 | 2077 | SDP | 45 | F | II | 125.33 | 122.00 | 5008.89 | 41.06 | 2042.44 | 16.74 |
| MPB | 09 | 2792 | DNW | 19 | F | III | 180.17 | 141.67 | 5476.00 | 38.65 | 2319.78 | 16.37 |
| AOS | 10 | 2535 | FAC | 42 | F | III | 154.44 | 156.33 | 5373.00 | 34.37 | 2522.22 | 16.13 |
| | | | Mean = | 46.80 | | | | | Mean = | 39.21 | | 16.95 |
| | | | SD = | 13.97 | | | | | SD = | 3.73 | | 1.25 |
| | | | n = | 10 | | | | | n = | 10 | | 10 |
| | | | | | | | | | SE = | 1.18 | | 0.40 |
| | | | | | | | | | t = | 1.83 | | 1.83 |
| | | | | | | | | | Labeled | 37 | | 16 |
| | | | | | | | | | SPF = | | | |
| | | | | | | | | | | | Mean: Valid (Y/N) | Yes |
| | | | | | | | | | | | Labeled: Valid | Yes |
| | | | | | | | | | | | (Y/N) | |

### Example 2

### Evaluation of Effectiveness of Eye Cream

A composition was prepared containing the following components:* * Components listed below the line are present in an amount of less than 1% by weight.

| Ingredients | % |
|---|---|
| Octinoxate | 7.5 |
| Zinc Oxide | 10.1 |
| Water | QS to 100 |
| Ethylhexyl Stearate | 5.2 |
| Cyclopentasiloxane | 4.5 |
| Propanediol | 4 |
| Potassium Cetyl Phosphate | 3 |
| Butylene Glycol | 3 |
| C₁₂₋₁₅ Alkyl Benzoate | 3 |
| Adipic Acid/Diethylene Glycol/Glycerin Crosspolymer | 2 |
| Isohexadecane | 1.8 |
| Cetearyl Alcohol | 1.14-1.54 |
| Glyceryl Stearate | 1.25 |
| PEG-100 Stearate | 1.25 |
| Mica | 0.9-1.098 |

Phenoxyethanol
Hydroxyethyl Acrylate/Sodium Acryloyldimethyl Taurate Copolymer
Imperata Cylindrica Root Extract
Titanium Dioxide
Ceteareth-20
Squalane
Glycerin
Triethoxycaprylylsilane
Xanthan Gum
Helianthus Annuus (Sunflower) Seed Oil
Polysorbate 60
Trisodium Ethylenediamine Disuccinate
Chlorphenesin
Citric Acid
Dimethicone
Dipotassium Glycyrrhizate
Ethylhexylglycerin
Oleth-3 Phosphate
PEG-8
Punica Granatum Seed Oil
Carthamus Tinctorius (Safflower) Seed Oil
Avena Sativa (Oat) Peptide
Silica
Camellia Sinensis Leaf Extract
Carbomer
Olea Europaea (Olive) Fruit Extract
Euterpe Oleracea Fruit Extract
Methylisothiazolinone
Tocotrienols
Lauroyl Lysine

The above composition had an SPF of about 40 and was evaluated in its effectiveness to: (1) reduce the appearance of fine line and wrinkles in the eye area; (2) improve skin hydration/moisturization; and (3) improve firmness.

Enrollment in Study

Panel selection was accomplished by advertisements in local periodicals, community bulletin boards, phone solicitation, electronic media, or any combination thereof. Informed consent was obtained from each volunteer prior to initiating the study describing reasons for the study, possible adverse effects, associated risks and potential benefits of the treatment and their limits of liability. Panelists signed and dated the informed consent document and a photography release form to indicate their authorization to proceed and acknowledge their understanding of the contents. Each subject was assigned a permanent identification number and completed an extensive medical history form.

Volunteers that were deemed suitable for inclusion in the study satisfied the following criteria:
1. Female
2. Caucasian
3. Ages 35-60
4. Individuals who were in good general health.
5. Individuals who were free of any dermatological or systemic disorder, which would interfere with the results, at the discretion of the Investigator.
6. Individuals who completed a preliminary medical history, HIPAA, and photography release.
7. Individuals who read, understood and signed an informed consent document.
8. Individuals who were able to cooperate with the Investigator and research staff, had test product applied according to the protocol, and completed the full course of the study.
9. Individuals who did not participate in any other clinical studies using the same test sites (face) in the past 15 days.
10. Individuals with mild/moderate/severe fine lines and wrinkles around the eye area.
11. Individuals who agreed to refrain from using all cosmetics and/or personal care products (i.e., soaps, creams, lotions) on their face with the exception of those products provided by the testing service for the duration of the study. Subjects were allowed to wear lipstick/lip-gloss, eyeliner and mascara on their face except for study day visits.
12. Individuals who agreed not to sunbathe/tan for the duration of the study.

Volunteers that were deemed unsuitable for inclusion in the study had the following criteria:
1. Individuals who had a history of acute or chronic disease that would likely interfere with or increase the risk on study participation.
2. Individuals with active (flaring) disease or chronic skin allergies (atopic dermatitis/eczema), or those who had recently undergone treatment for skin cancer (within the last 12 months).
3. Individuals with damaged skin in close proximity to the test site (face), e.g., sunburn, tattoos, scars, piercings or other disfigurations.
4. Individuals who had any history, which, in the Investigator's opinion, indicated the potential for harm to the subject or placed the validity of the study in jeopardy.
5. Female volunteers who indicated that they were pregnant, planning a pregnancy or nursing.
6. Individuals who treated their diabetes with injectable insulin.
7. Individuals who had any medical procedure, such as laser resurfacing, or plastic surgery to the test site (face) within the last 12 months. This included Botox®, Restylane®, collagen or other cosmetic filling procedures.
8. Individuals who were currently using or during the last 3 months had used, Retin A, or other Rx/OTC Retinyl A, hydroquinone (skin lightening) or other astringent derived products or alpha hydroxyl acid treatments for photo-aging and fine lines/wrinkles.
9. Individuals with a known history of hypersensitivity to any cosmetics and/or personal care products.
10. Individuals who were employees of the testing service.

### Experimental Technique

Bioinstrumental Method to Measure Water Content of Human Skin

Changes in skin conductance, impedance or capacitance were used to study epidermal hydration in vivo. The measurement was made on the difference in dielectric constant. Skin has a low dielectric constant and water has a high dielectric constant of 81. When skin is hydrated, conductance and capacitance increases and impedance decreases. The measuring capacitor shows changes in capacitance according to the moisture content of the tissue. A glass lamina separates the metallic tracks in the probe head from the skin in order to prevent current conduction in the tissue. An electric scatter field penetrates the skin during the measurement and the dielectricity is determined.

Corneometer® CM 825 (Courage and Khazaka, Germany) was used to measure the electrical capacitance/hydration of the skin. Three replicate measurements were taken on each test site at each time point.

Bioinstrumental Method to Measure Firmness of Human Skin

The biomechanical properties of human skin are the result of a complex combination of elastic (elastin fibers) and viscous (collagen fibers and surrounding intercellular ground substance) components. A Cutometer® allows the measurement of the viscoelastic properties of the skin in vivo. The measuring principle of the Cutometer® is based on suction. A defined negative air pressure is created and applied on the skin surface through the opening of a probe drawing the skin into its aperture. The resulting vertical deformation of the skin is measured by determining the depth of skin penetration into the probe. This is achieved by a noncontact optical system consisting of a light transmitter and a light recipient. Two glass prisms project the light from transmitter to recipient, where the diminution of the infrared light beam depending on the penetration depth of the skin is measured.

Cutometer® MPA 580 (Courage and Khazaka, Germany) was used to measure skin firmness. The results provided the average percent change in skin firmness from baseline and percent of subjects showing improvement.

Clinical Photography for Visual Evaluation of Fine Lines and Wrinkles in the Eye Area

Generally, no distinction is made between fine lines and wrinkles in visual grading using clinical photography. Photographs were taken in accordance with regulations provided by consumer protection agencies such as the Federal Trade Commission, the Food and Drug Administration and several other regulatory authorities. The following guidelines were followed: 1) Head position is the same in before and after photos, 2) Same lighting conditions are used and the distance from the camera is same for both, before and after picture, and 3) Same room and background is used for both before and after picture.

Clinical photographs of subjects' faces (frontal, left lateral and right lateral) were taken and evaluated with Canfield's VISIA CR system using the standard 1 mode. Photographs obtained were evaluated for fine lines and wrinkles in the eye area using a 0-9 scale category, where 0=None, 1-3=Mild, 4-6=Moderate, and 7-9=Severe.

The results provided percent of subjects showing improvement from baseline.

Measurement of Fine Lines and Wrinkles Using Silicone Replicas

The measurement was achieved by obtaining a topical 3D micro-anatomical profilometry via silicone replicas. Generally, sites evaluated for fine lines and wrinkles include the periocular areas located on the side of the eyes (Crow's feet). Two (2)×two (2) cm adhesive templates were affixed to the test site and Replifo vinyl silicone (Cuderm Corporation, Dallas, Tex.) was dispensed onto the template demarcated areas. After about 5 minutes, the replicas were cured and gently removed from the skin surface. Image analysis using a Cohu solid state B&W camera, 50 mm lens/30 mm extension, Coreco TCI Ultra frame grabber interfaced with an IBM compatible PC was conducted by Cudenn Corporation, Dallas, Tex.

Specifically during the image analysis phase, a collimated light source was directed at a 25-degree angle from the plane of the replica. The replica was gently placed in the holder and was rotated to align for normal or parallel exposure to the incident light direction. Further changes in the gradient of light intensity can produce changes in luminance, which in turn is used to assess changes in skin roughness displayed by the replica. The normal sampling orientation provides texture measurements sensitive to the major expression-induced lines or “coarse wrinkles” and the parallel sampling orientation provided texture measurements sensitive to the minor fine lines or “fine lines”.

The shadow texture produced by the oblique lighting of the negative replica was analyzed by two types of assay methods:
1. Measuring the luminance along a set of 10 equal length parallel lines running across the replica parallel to the lighting direction. The variance in luminance was treated as indicative of the roughness and analyzed by traditional surface roughness statistics.
2. The replica image area was divided into 10 equal width bands and the shadow like features were detected according to their luminance values.

The below 7 wrinkle texture parameters measure various aspects of the image produced by the replica surface:
1. Rz=skin roughness texture
2. Ra=skin roughness texture
3. IDL=increases with roughness of the surface
4. Spacing=mean distance between adjacent strong shadow features
5. Breadth=proportional to the depth of the wrinkle producing the shadow
6. Shadows=relative area of shadows cast by all the wrinkles and fine lines
7. NumWr=total number of shadowy features available to calculate spacing and breadth

The results provide average percent change for each parameter from baseline and percent of subjects showing improvement. The results were reported for both fine lines and coarse wrinkles.

Self-Assessment Questionnaire

Each subject was instructed to complete a self-assessment questionnaire at the Week 4 and Week 8 post-treatment interval.

Procedure

Subjects reported to the facility a minimum of five (5) days prior to the start of the study. Subjects completed an informed consent form, medical history form, photo release, and HIPAA form prior to beginning all study related activities.

Five (5) days prior to the start of the study, subjects began the washout period. Subjects received a neutral soap (Neutrogena®) to use for washing their face during the washout period and study period. Subjects were given specific instructions prohibiting use of any cosmetics and personal care products, (i.e., lotions, soap, cleansers, and make-up) with exception of those provided by the testing service and lipstick/lip-gloss, eyeliner and mascara during the study period.

Following the washout period, subjects returned to the facility for baseline measurements of the face. Subjects were instructed to cleanse their face with a neutral soap and gently pat dry with a paper towel.

Thereafter, subjects remained quietly seated for a minimum of fifteen (15) minutes in a room maintained at approximately 20-24° C. and approximately 30%-50% relative humidity. Temperature and humidity were recorded during subject testing.

Subjects had the following evaluations performed by a trained staff member:

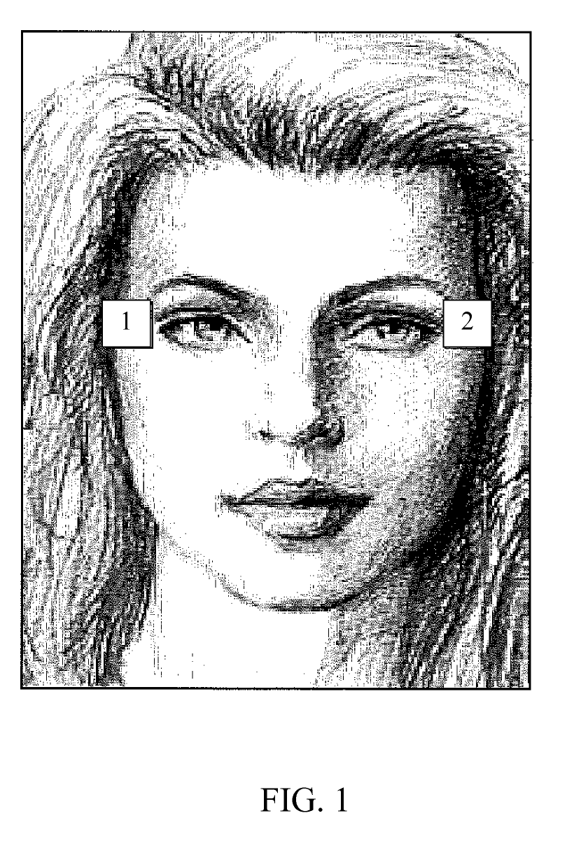

Baseline (Pre-Treatment):
1. Close-up facial photography (frontal, left lateral and right lateral)
2. Skin hydration at sites 1 and 2 (See FIG. 1)
3. Skin firmness at sites 1 and 2
4. Silicone replicas at sites 1 and 2

The test product was applied on the face by the subject. Using gentle pressure, the product was applied by gliding a cooling metal applicator under the eye, starting at the inner corner and moving outward. The subject had the below procedures/measurements performed by trained staff 10 minutes (±2 min) post-treatment:

10 Minute (Post-Treatment):
Skin hydration at sites 1 and 2

Subjects were given the test products along with product use instructions for at home use for 8 weeks. Subjects were instructed to apply the test product twice daily (morning and night). At Week 4 (±3 days) of treatment with the test product, subjects were instructed to return to the testing facility with their test products. Test products were weighed for compliance. Subjects were instructed not to use any test product on the day of their scheduled visit.

Subjects were instructed to cleanse their face with a neutral soap and gently pat dry with a paper towel. Thereafter, subjects remained quietly seated for a minimum of fifteen (15) minutes in a room maintained at approximately 20-24° C. and approximately 30%-50% relative humidity. Temperature and humidity were recorded during subject testing.

The following evaluations were made on their face at the Week 4 post treatment visit by a trained staff member:

4 Weeks (Post-Treatment):
1. Close-up facial photography (frontal, left lateral and right lateral)
2. Skin hydration at sites 1 and 2
3. Skin firmness at sites 1 and 2
4. Silicone replicas at sites 1 and 2
5. Post-treatment questionnaire

At Week 8 (±3 days) of treatment with the test product, subjects were instructed to return to the testing facility with their test products. Test products were weighed for compliance. Subjects were instructed not to use any test product on the day of their scheduled visit.

Subjects were instructed to cleanse their face with a neutral soap and gently pat dry with a paper towel. Thereafter, subjects remained quietly seated for a minimum of fifteen (15) minutes in a room maintained at approximately 20-24° C. and approximately 30%-50% relative humidity. Temperature and humidity were recorded during subject testing.

The following evaluations were made on their face at the Week 8 post treatment visit by a trained staff member:

8 Weeks (Post-Treatment):
1. Close-up facial photography (frontal, left lateral and right lateral)
2. Skin hydration at sites 1 and 2
3. Skin firmness at sites 1 and 2
4. Silicone replicas at sites 1 and 2
5. Post-treatment questionnaire

Subjects returned any remaining test product at this visit. Subjects were dismissed from the study upon completion of Week 8 measurements.

### Study Results and Analysis

A total of 20 healthy Caucasian female subjects consented, enrolled, and completed the clinical study. There were no adverse events reported during the 4 weeks of the study period. The results of the study are provided below.

Skin Hydration by Corneometer®

TABLE 1
Mean Skin Hydration Values

| Interval | Mean | SD |
|---|---|---|
| Baseline | 35.21 | 8.66 |
| 10 Minutes | 56.81 | 13.49 |
| Week 4 | 41.15 | 8.18 |
| Week 8 | 48.05 | 7.95 |

TABLE 2
Descriptive Statistics of Skin Hydration Differences from Baseline

| Interval | Parameter | Skin Hydration Differences from Baseline |
|---|---|---|
| 10 Minutes | Mean | 21.60 |
| | SD | 9.34 |
| | % Change | 61.33% |
| | p | ≦0.001 |
| | % Improvers | 100.00% |
| | p | ≦0.001 |
| Week 4 | Mean | 5.94 |
| | SD | 8.58 |
| | % Change | 16.86% |
| | p | 0.006 |
| | % Improvers | 70.00% |
| | p | NS |
| Week 8 | Mean | 12.83 |
| | SD | 8.58 |
| | % Change | 36.45% |
| | p | ≦0.001 |
| | % Improvers | 100.00% |
| | p | ≦0.001 |

NS = Not Significant
BOLD values indicate statistical significance (p ≦ 0.05)

A positive difference indicates improvement in skin hydration. There was a significant improvement after 10 minutes and at Week 8 seen in all test subjects. At Week 4, 70% of the subjects demonstrated an improvement. The results demonstrate a statistically significant improvement in skin hydration from baseline at 10 minutes, 4 weeks and 8 weeks post-treatment. A statistically significant number of subjects showed improvement in skin hydration at 10 minutes and 8 weeks post-treatment.

Skin Firmness by Cutometer®

TABLE 3
Mean Skin Firmness Values

| Interval | Mean | SD |
|---|---|---|
| Baseline | 0.4499 | 0.0709 |
| Week 4 | 0.4335 | 0.0962 |
| Week 8 | 0.4545 | 0.0853 |

TABLE 4
Descriptive Statistics of Skin Firmness Differences from Baseline

| Interval | Parameter | Skin Firmness Differences from Baseline |
|---|---|---|
| Week 4 | Mean | −0.0164 |
| | SD | 0.0784 |
| | % Change | −3.65% |
| | p | NS |
| | % Improvers | 61.90% |
| | p | NS |
| Week 8 | Mean | 0.0046 |
| | SD | 0.0551 |
| | % Change | 1.02% |
| | p | NS |
| | % Improvers | 47.62% |
| | p | NS |

NS = Not Significant

A negative difference indicates improvement in skin firmness. There was a slight improvement in skin firmness in Week 4, but no improvement was seen in Week 8.

Fine Lines and Wrinkles of the Periocular Area Using Visual Grading of Clinical Photography

TABLE 5
Mean Fine Lines and Wrinkles Scores From Visual Grading
SCALE: 0 = None, 1-3 = Mild, 4-6 = Moderate, 7-9 Severe

| Interval | Mean | SD |
|---|---|---|
| Baseline | 5.75 | 1.59 |
| Week 4 | 5.49 | 1.71 |
| Week 8 | 5.39 | 1.69 |

TABLE 6
Descriptive Statistics of Visual Grading
Score Differences from Baseline

| Interval | Parameter | Skin Hydration Differences from Baseline |
|---|---|---|
| Week 4 | Mean | −0.26 |
| | SD | 0.31 |
| | % Change | −4.57% |
| | p | 0.001 |
| | % Improvers | 65.00% |
| | p | NS |
| Week 8 | Mean | −0.36 |
| | SD | 0.46 |
| | % Change | −6.30% |
| | p | 0.002 |
| | % Improvers | 75.00% |
| | p | NS |

NS = Not Significant
BOLD values indicate statistical significance (p ≦ 0.05)

Negative differences indicate improvements in the appearance of fine lines and wrinkles of the periocular area. There was a statistically significant improvement in the appearance of fine lines and wrinkles of the periocular area at 4 weeks and 8 weeks post-treatment.

Fine Lines and Wrinkles of the Crow's Feet Area Using Silicone Replica

TABLE 7
Mean Silicone Replica Data for Coarse Wrinkles

| | Interval | | |
|---|---|---|---|
| | Baseline | Week 4 | Week 8 |
| Parameter | (Mean ± SD) | (Mean ± SD) | (Mean ± SD) |
| Rz | 166.9 ± 26.9 | 168.0 ± 38.3 | 175.6 ± 33.9 |
| Ra | 36.9 ± 8.8 | 39.2 ± 12.4 | 40.4 ± 11.4 |
| IDL | 7.0 ± 1.4 | 7.3 ± 2.2 | 7.6 ± 2.0 |
| Shadows | 12.4 ± 5.2 | 11.8 ± 7.8 | 12.9 ± 5.7 |
| NumWr | 106.2 ± 38.2 | 91.0 ± 39.1 | 103.1 ± 37.5 |
| Spacing | 1.1 ± 0.5 | 1.2 ± 0.5 | 1.1 ± 0.4 |
| Breadth | 0.3 ± 0.1 | 0.3 ± 0.1 | 0.3 ± 0.1 |

TABLE 8
Descriptive Statistics of Silicone Replica Data for Coarse Wrinkles from Baseline

| | | Silicone Replica Values from Baseline | | | | | | |
|---|---|---|---|---|---|---|---|---|
| Interval | Parameter | Rz | Ra | IDL | Shadows | NumWr | Spacing | Breadth |
| Week 4 | Mean | 1.1 | 2.3 | 0.3 | −0.6 | −15.2 | 0.1 | 0.0 |
| | SD | 34.8 | 12.1 | 1.8 | 6.6 | 33.6 | 0.5 | 0.1 |
| | % Change | −0.61% | 6.22% | 3.73% | −4.99% | −14.30% | 7.87% | 0.41% |
| | p | NS | NS | NS | NS | 0.058 | NS | NS |
| | % | 45.00% | 50.00% | 60.00% | 60.00% | 70.00% | 60.00% | 50.00% |
| | Improvers | | | | | | | |
| | p | NS | NS | NS | NS | NS | NS | NS |
| Week 8 | Mean | 8.7 | 3.5 | 0.6 | 0.5 | −3.1 | 0.0 | 0.0 |
| | SD | 32.1 | 10.2 | 1.6 | 5.5 | 31.2 | 0.5 | 0.1 |
| | % Change | 5.23% | 9.51% | 9.14% | 3.99% | −2.92% | −0.89% | 4.35% |
| | p | NS | NS | 0.094 | NS | NS | NS | NS |
| | % | 35.00% | 35.00% | 40.00% | 55.00% | 55.00% | 55.00% | 40.00% |
| | Improvers | | | | | | | |
| | p | NS | NS | NS | NS | NS | NS | NS |

NS = Not Significant
Italicized values indicate directional significance (p ≦ 0.10)

Rz, Ra, IDL, Shadows, and NumWr are parameters that indicate skin smoothness. Negative differences of Rz, Ra, IDL, Shadows, NumWr, and Breadth indicate improvement and an increase in skin smoothness. There was an increase in skin smoothness of wrinkles at 4 weeks post-treatment, but it was not statistically significant.

Spacing is a parameter that indicates the number of fine lines or wrinkles. An increase in spacing (positive difference from baseline) indicates a decrease in the number of wrinkles or fine lines. There was a decrease in the number of wrinkles at 8 weeks post-treatment, but it was not statistically significant.

Breadth is a parameter that indicates the depth of fine lines or wrinkles. A decrease in breadth (negative difference from baseline) indicates a decrease in the depth of wrinkles or fine lines.

TABLE 9
Mean Silicone Replica Data for Fine Lines

Interval

| | Baseline | Week 4 | Week 8 |
|---|---|---|---|
| Parameter | (Mean ± SD) | (Mean ± SD) | (Mean ± SD) |
| Rz | 120.0 ± 23.0 | 114.4 ± 24.0 | 109.1 ± 26.7 |
| Ra | 25.1 ± 4.7 | 24.5 ± 5.3 | 23.2 ± 5.5 |
| IDL | 5.2 ± 1.4 | 4.9 ± 1.2 | 4.5 ± 1.3 |
| Shadows | 4.9 ± 2.7 | 4.4 ± 2.8 | 4.8 ± 3.1 |
| NumWr | 64.0 ± 36.6 | 50.7 ± 31.0 | 59.5 ± 38.1 |
| Spacing | 1.6 ± 0.5 | 2.0 ± 0.8 | 1.9 ± 0.8 |
| Breadth | 0.2 ± 0.0 | 0.2 ± 0.0 | 0.2 ± 0.0 |

TABLE 10
Descriptive Statistics of Silicone Replica Data for Fine Lines from Baseline

| | | Silicone Replica Values Baseline | | | | | | |
|---|---|---|---|---|---|---|---|---|
| Interval | Parameter | Rz | Ra | IDL | Shadows | NumWr | Spacing | Breadth |
| Week 4 | Mean | −5.6 | −0.6 | −0.4 | −0.5 | −13.3 | 0.4 | 0.0 |
| | SD | 16.2 | 4.3 | 0.9 | 2.4 | 23.4 | 0.7 | 0.0 |
| | % Change | −6.77% | −2.53% | −7.39% | −10.47% | −20.74% | 24.87% | 5.74% |
| | p | NS | NS | 0.074 | NS | 0.02 | 0.019 | NS |
| | % | 65.00% | 55.00% | 75.00% | 60.00% | 80.00% | 70.00% | 30.00% |
| | Improvers | | | | | | | |
| | p | NS | NS | NS | NS | 0.047 | NS | NS |
| Week 8 | Mean | −10.9 | −1.9 | −0.7 | −0.1 | −4.5 | 0.4 | 0.0 |
| | SD | 19.1 | 4.4 | 0.9 | 2.4 | 25.8 | 0.8 | 0.0 |
| | % Change | −9.06% | −7.59% | −13.28% | −1.73% | −6.99% | 22.51% | 7.26% |
| | p | 0.02 | 0.065 | 0.004 | NS | NS | NS | 0.035 |
| | % | 65.00% | 65.00% | 70.00% | 55.00% | 55.00% | 55.00% | 30.00% |
| | Improvers | | | | | | | |
| | p | NS | NS | NS | NS | NS | NS | NS |

NS = Not Significant
BOLD values indicate statistical significance (p ≦ 0.05)
Italicized values indicate directional significance (p ≦ 0.10)

Rz, Ra, IDL, Shadows, and NumWr are parameters that indicate skin smoothness. Negative differences of Rz, Ra, IDL, Shadows, NumWr, and Breadth indicate improvement and an increase in skin smoothness. There was an increase in skin smoothness of fine lines at Week 4 post-treatment, but it was not statistically significant. There was a statistically significant increase in skin smoothness of fine lines at Week 8 post-treatment.

Spacing is a parameter that indicates the number of fine lines or wrinkles. An increase in spacing (positive difference from baseline) indicates a decrease in the number of wrinkles or fine lines. There was a statistically significant decrease in the number of fine lines at 4 weeks post-treatment.

Analysis of Self-Assessment Questionnaire

### SCALE: 1=Strongly Agree, 2=Agree, 3=Neutral, 4=Disagree, 5=Strongly Disagree

| | | Percentage of Subjects (%) Agreeing | |
|---|---|---|---|
| Post-Treatment Question | | Week 4 | Week 8 |
| 1. | My skin feels moisturized. | 75.00% | 90.00% |
| 2. | My skin feels smoother. | 80.00% | 85.00% |
| 3. | The fine lines/wrinkles | 70.00% | 70.00% |
| | around my eyes are less | | |
| | noticeable. | | |
| 4. | This product reduced the | 65.00% | 70.00% |
| | look of dark circles under | | |
| | my eyes. | | |
| 5. | This product made my | 80.00% | 80.00% |
| | eyes look refreshed. | | |
| 6. | This product made my eye | 70.00% | 75.00% |
| | area look more youthful. | | |
| 7. | This product made my eye | 65.00% | 75.00% |
| | area look more rested. | | |
| 8. | There is a reduction in the | 65.00% | 65.00% |
| | appearance of fine lines/ | | |
| | wrinkles. | | |
| 9. | This product evens out my | 65.00% | 65.00% |
| | skin tone. | | |
| 10. | This product helps my | 75.00% | 75.00% |
| | skin looks younger. | | |
| 11. | This product improves | 80.00% | 75.00% |
| | skin texture. | | |
| 12. | My skin looks firmer. | 80.00% | 75.00% |
| 13. | I see a positive change to | 65.00% | 65.00% |
| | my skin immediately upon | | |
| | application. | | |
| 14. | This product has improved | 75.00% | 75.00% |
| | the look of my skin. | | |
| 15. | My skin looks radiant | 65.00% | 70.00% |
| 16. | I saw an improvement in | 75.00% | 75.00% |
| | skin elasticity | | |

BOLD type percentages indicate statistical significance (p ≦ 0.05)

The results of the questionnaire demonstrate that subjects noticed a marked improvement in their skin's appearance and texture. Thus, it appears that on both an objective and a subjective basis, there was a surprising and unexpected increase in skin hydration, reduction in appearance of fine lines and wrinkles in an eye area, decrease in the number of fine lines of a crow feet's area, improvement in skin texture or smoothness of fine lines of a crow feet's area, or a combination thereof in a statistically significant percentage of test subjects.

The term “about,” as used herein, should generally be understood to refer to both numbers in a range of numerals. Moreover, all numerical ranges herein should be understood to include each whole integer, and preferably each number within the range (e.g., 0.9 to 1.2 would include 1 and 1.1, as well).

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those components, amounts, and methods shown and described herein will become apparent to those of ordinary skill in the art and are intended to fall within the scope of the appended claims.

What is claimed is:

1. A photoprotectant cosmetic composition adapted to application on a user's skin comprising:
a sunscreen component comprising: (i) octinoxate; and (ii) zinc oxide, each present in an amount sufficient to collectively provide ultraviolet A and B protection with an SPF of at least about 10;
a mica component comprising mica in an amount sufficient to reduce or eliminate an appearance of dark skin; and
an oat peptide component in an amount sufficient to reduce or eliminate at least the appearance of fine lines and wrinkles on the user's skin.

2. The photoprotectant cosmetic composition of claim 1, formulated as a cream.

3. The photoprotectant cosmetic composition of claim 1, formulated to be an emulsion.

4. The photoprotectant cosmetic composition of claim 1, wherein the sufficient amount of octinoxate is about 5% to about 15% by weight of the composition and the zinc oxide is about 5% to about 25% by weight of the composition, the sufficient amount of mica component is about 0.5% to about 1.5% by weight of the composition, and the sufficient amount of the oat peptide component is about 0.02% to about 0.15% by weight of the composition.

5. The photoprotectant cosmetic composition of claim 4, wherein the octinoxate is present in an amount from about 8% to about 15% by weight of the composition, the zinc oxide is present in an amount from about 5% to about 25% by weight of the composition, the mica component is present in an amount from about 0.8% to about 1.2% by weight of the composition, and the oat peptide component is present in the composition in an amount from about 0.05% to about 0.1% by weight of the composition.

6. The photoprotectant cosmetic composition of claim 4 that does not include octocrylene or amiloxate.

7. The photoprotectant cosmetic composition of claim 4, wherein the SPF of the composition is from about 10 to about 50.

8. The photoprotectant cosmetic composition of claim 4, wherein the SPF of the composition is from about 35 to about 50.

9. The photoprotectant cosmetic composition of claim 1, which further comprises:
a stearate component;
an alcohol component; and
a formulating agent comprising cyclopentasiloxane, alkyl benzoate, potassium cetyl phosphate, an adipic acid/diethylene glycol/glycerin crosspolymer, or isohexadecane, or a combination of one or more thereof.

10. The photoprotectant cosmetic composition of claim 9, wherein the stearate component comprises ethylhexyl stearate, glyceryl stearate, or PEG-100 stearate, or a combination thereof, the alcohol component comprises a diol, a glycol, or cetearyl alcohol or a combination thereof, and the alkyl benzoate is a C₁₂₋₁₅ alkyl benzoate.

11. The photoprotectant cosmetic composition of claim 10, wherein the stearate component comprises a combination of ethylhexyl stearate, glyceryl stearate, and PEG-100 stearate, the alcohol comprises a combination of propanediol, butylene glycol, and cetearyl alcohol, and the formulating agent comprises a combination of cyclopentasiloxane, alkyl benzoate, potassium cetyl phosphate, an adipic acid/diethylene glycol/glycerin crosspolymer, and isohexadecane.

12. The photoprotectant cosmetic composition of claim 9, wherein the stearate component is present in an amount of from about 6% to about 7.5% by weight of the composition, the alcohol is present in an amount of from about 8% to about 9% by weight of the composition, and the formulating agent is present in an amount of from about 5.5% to about 11% by weight of the composition.

13. A photoprotectant cosmetic composition adapted to application on a user's skin comprising:
a sunscreen component consisting of: (i) octinoxate; and (ii) zinc oxide, each present in an amount sufficient to collectively provide ultraviolet A and B protection with an SPF of at least about 10;
a mica component comprising mica in an amount sufficient to reduce or eliminate the appearance of dark skin adjacent an eye; and
an oat peptide component in an amount sufficient to reduce or eliminate at least the appearance of fine lines and wrinkles on the user's skin,
where the composition does not include a detectable amount of octocrylene and amiloxate.

14. The photoprotectant cosmetic composition of claim 12, wherein the sufficient amount of octinoxate is about 5% to about 15% by weight of the composition and the zinc oxide is about 5% to about 25% by weight of the composition, the sufficient amount of mica component is about 0.5% to about 1.5% by weight of the composition, and the sufficient amount of the oat peptide component is about 0.02% to about 0.15% by weight of the composition.

15. A method of improving the aesthetic appearance of skin comprising topically applying a composition in an amount effective to improve the aesthetic appearance of the skin, the composition comprising:
a sunscreen component comprising: (i) octinoxate; and (ii) zinc oxide;
a mica component; and
an oat peptide component.

16. The method of claim 15, wherein the composition further comprises:
a stearate component;
an alcohol component; and
a formulating agent comprising cyclopentasiloxane, alkyl benzoate, potassium cetyl phosphate, an adipic acid/diethylene glycol/glycerin crosspolymer, or isohexadecane, or a combination of one or more thereof.

17. The method of claim 15, wherein the improvement in aesthetic appearance is selected from the group consisting of an increase in skin hydration, reduction in appearance of fine lines and wrinkles in an eye area, decrease in the number of fine lines of a crow feet's area, improvement in skin texture or smoothness of fine lines of a crow feet's area, or a combination thereof.

18. The method of claim 17, wherein the improvement in aesthetic appearance is an increase in skin hydration, and topical application effects an increase in skin hydration of greater than 10% from baseline.

19. The method of claim 17, wherein the improvement in aesthetic appearance is a reduction in appearance of fine lines and wrinkles in an eye area, and topical application effects a reduction of greater than 3% from baseline.

20. The method of claim 17, wherein the improvement in aesthetic appearance is an improvement in skin texture or smoothness of fine lines of a crow feet's area, and topical application effects an increase of smoothness of greater than 5% from baseline.

21. The method of claim 17, wherein the improvement in aesthetic appearance is a decrease in the number of fine lines of a crow feet's area and topical application effects a decrease in the number of fines lines of greater than 20% from baseline.

22. The method of claim 17, wherein the topical applying occurs once or twice daily for about four to eight weeks.
